(19) United States
(12) Patent Application Publication
(10) Pub. No.: US 2021/0244738 A1
(43) Pub. Date: Aug. 12, 2021

(54) COMPOSITIONS AND METHODS FOR SUPPRESSING NONSENSE MUTATIONS

(71) Applicants: The UAB Research Foundation, Birmingham, AL (US); Southern Research Institute, Birmingham, AL (US)

(72) Inventors: David M. Bedwell, Birmingham, AL (US); Kim M. Keeling, Birmingham, AL (US); Ming Du, Birmingham, AL (US); Steven M. Rowe, Birmingham, AL (US); Venkateshwar Mutyam, Hoover, AL (US); Amna Siddiqui, Birmingham, AL (US); James Robert Bostwick, Birmingham, AL (US)

(21) Appl. No.: 16/972,148

(22) PCT Filed: Jun. 14, 2019
(86) PCT No.: PCT/US19/37186

§ 371 Date: Dec. 4, 2020

Related U.S. Application Data
(60) Provisional application No. 62/684,931, filed on Jun. 14, 2018.

(51) Int. Cl. A61K 31/519; C12Q 1/6883

(52) U.S. Cl. CPC A61K 31/519; C12Q 2600/156; C12Q 1/6883

(57) ABSTRACT

Disclosed herein are methods of treating or preventing a disease caused by nonsense mutations, or ameliorating one or more symptoms associated therewith, that involve administering to a patient in need thereof a therapeutically or prophylactically effective amount of triamterene, or a pharmaceutically acceptable salt, hydrate, solvate, clathrate, prodrug, or polymorph thereof.

### CROSS-REFERENCE TO RELATED APPLICATIONS

This application claims benefit of U.S. Provisional Application No. 62/684,931, filed Jun. 14, 2018, which is hereby incorporated herein by reference in its entirety.

### SEQUENCE LISTING

222104-2870 Sequence Listing_ST25″ created on Jun. 10, 2019. The content of the sequence listing is incorporated herein in its entirety.

### BACKGROUND

Many human genetic disorders result from nonsense mutations, where one of the three stop codons (UAA, UAG or UGA) replaces an amino acid-coding codon, leading to premature termination of translation and eventually to truncated, inactive proteins. Currently, hundreds of such nonsense mutations are known, and several were shown to account for certain cases of fatal diseases, including, for example, cystic fibrosis (OF), Duchenne muscular dystrophy (DMD), ataxia-telangiectasia, Hurler syndrome, hemophilia A, hemophilia B, Tay-Sachs, Rett Syndrome, Usher Syndrome, Severe epidermolysis bullosa and more. For many of those diseases there is presently no effective treatment.

### SUMMARY

Disclosed herein are methods of treating or preventing a disease caused by nonsense mutations, or ameliorating one or more symptoms associated therewith, that involve administering to a patient in need thereof a therapeutically or prophylactically effective amount of triamterene, or a pharmaceutically acceptable salt, hydrate, solvate, clathrate, prodrug, or polymorph thereof.

The details of one or more embodiments of the invention are set forth in the accompanying drawings and the description below, Other features, objects, and advantages of the invention will be apparent from the description and drawings, and from the claims.

### DESCRIPTION OF DRAWINGS

FIGS. 1A to 1C identify of compounds that can suppress termination at nonsense mutations. FIG. 1A is a schematic of the W134X NanoLuc reporter, which was stably expressed in Fischer rat thyroid cells to screen 10,000 low molecular weight compounds for those that induce readthrough, inhibit NMD, or both. FIG. 1B shows Nanoluc activity measured in cells treated with each compound (at either 60 μM or 30 μg/ml) relative to the Nanoluc activity measured in untreated cells (basal). FIG. 1C shows NanoLuc activity in cells treated with each compound (at either 60 μM or 30 μg/ml) in combination with the readthrough compound, G418 (100 μg/ml). Each column represents the average NanoLuc activity obtained from two independent screens. The lines indicate the Nanoluc activity generated in the G418 positive control.

FIG. 2 shows the ability of hit compounds to restore α-L-iduronidase activity in Idua-W402X mouse embryonic fibroblasts (MEFs). Immortalized MEFs that are homozygous for the Idua-W402X nonsense mutation were cultured in the presence of the most active hits from the luciferase screen (Table 1). The MEFs were cultured in the presence of vehicle alone or in the presence of a compound (60 μM or 30 μg/ml) for 48 hours. α-L-iduronidase activity was measured in cell lysates using a 4-methyl-umbellyferyl (FMU)-iduronide fluorescent substrate and expressed as the nanomoles of FMU released per milligram of total cellular protein per hour. G418 is a potent, but toxic, readthrough compound that was used as a positive control. Each column represents the mean+/−SD of a representative experiment performed in quadruplicate. The top line corresponds to 0.3% of wildtype α-L-iduronidase activity, which is the amount of α-L-iduronidase activity required for phenotypic improvements in Idua-W402X mice. The bottom line indicates basal α-L-iduronidase activity in Idua-W402X MEFs, and the middle line indicates the α-L-iduronidase activity restored when MEFs are treated with G418.

FIGS. 3A and 3B shows triamterene and doxazocin increase α-L-iduronidase activity in Idua-W402X MEFs in a dose-dependent manner. FIG. 3A shows α-L-iduronidase activity restored in Idua-W402X MEFs when treated with triamterene alone or combined with G418 or PTC124 at the concentrations indicated. FIG. 3B shows α-L-iduronidase activity restored in Idua-W402X MEFs treated with doxazocin alone or combined with G418 or PTC124 at the concentrations indicated. Each column represents the mean+/−SD of the assay performed in quadruplicate. The data shown is representative data from one experiment. The top line represents 0.3% of wildtype enzyme activity. The middle line indicates the enzyme activity generated by the G418 positive control. The bottom line represents the residual enzyme activity in cells treated with vehicle only.

FIG. 4 shows triamterene and doxazocin decrease glycosaminoglycan storage in Idua-W402X MEFs in a dose-dependent manner. Total sulfated glycosaminoglycan (GAG) levels were quantitated in Idua-W402X MEFs using a dye-binding assay (Blyscan) and normalized to total cellular protein. Each column represents the mean+/−SD of a representative experiment performed in quadruplicate. The top line indicates the GAG levels in mutant cells treated with vehicle only. The bottom line indicates GAG levels in wildtype cells.

FIGS. 5A and 5B shows the ability of triameterene and doxazocin to readthrough a PTC in HEK293 cells. FIG. 5A shows dual luciferase reporters containing the W402X nonsense mutation (UAG) in the context of 3 codons of flanking upstream and downstream IDUA sequence were stably expressed in HEK293 cells. FIG. 5B shows reporter cells treated with triamterene or doxazocin for 24 hours and then assayed for firefly and Renilla luciferase activities, which were compared to the activities generated by the wildtype control. G418 served as a positive readthrough control. Each column represents the mean+/−SD of a representative experiment in quadruplicate.

FIG. 6 shows the effect of triamterene and doxazocin on steady-state Idua mRNA. Wildtype and Idua-W402X MEFs were cultured in the presence of triamterene (TRI) or doxazocin (DOX) for 48 hours. Total RNA was purified and reverse transcribed to cDNA. gPCR was performed to determine the Idua mRNA levels in wildtype and Idua-W402X MEFs that were treated with compound relative to those treated with vehicle only. Gapdh was used as a qPCR normalization control. Each column represents the mean+/−SD of a representative experiment performed with 6 replicates. The top line indicates the Idua level in control wildtype MEFs. The bottom line indicates the Idua level in control Idua-W402X MEFs.

FIGS. 7A to 7E show triamterene works predominantly through a PTC-dependent mechanism. Immortalized MEFs from an MPS I-H knockout (KO) mouse model that contains an insertional mutation not amenable to readthrough were cultured in the presence of triamterene for 48 hours and then assayed for α-L-iduronidase activity (FIG. 7A) and sulfated GAG levels (FIG. 7B). The line represents the value from the KO MEFs treated with the highest triamterene dose. FIGS. 7C to 7D show the amiloride, a diuretic that similarly to triamterene, targets the epithelial sodium channel (ENaC). Idua-W402X MEFs were cultured in the presence of amiloride at the indicated concentrations for 48 hours and then assayed for α-L-iduronidase activity (FIG. 7C) and sulfated GAG levels (FIG. 7D). The line in FIG. 7C indicates 0.3% of wild-type α-L-iduronidase activity. The line in FIG. 7D indicates the wild-type GAG level. FIG. 7E shows that thiazide, another diuretic that is often administered in conjunction with triamterene to control hypertension, does not affect the ability of triamterene to restore α-L-iduronidase activity. Idua-W402X MEF were treated with either thiazide, triamterene, or a combination of the two compounds as indicated by 48 hours and then assayed for α-L-iduronidase activity (FIG. 7E). The line represents 0.3% of wild-type α-L-iduronidase activity. For each panel in FIG. 7, each column represents the mean+/−SD of a representative experiment performed in quadruplicate.

FIGS. 8A to 8G show GAG levels in Idua-W402X mice after treatment with triamterene. 10-week-old Idua-W402X mice were orally administered triamterene once daily at the indicated dose. After two weeks, the mice were perfused and the tissues collected. Sulfated GAG levels were quantified in the indicated mouse tissues using a dye-binding assay (Blyscan) and normalized to the mass of the defatted, dried tissue. Each symbol represents the mean GAG value from an individual mouse. The bars indicate the mean+/−SD for each cohort. Exact p values calculated from the t-test are shown above the brackets, which indicate the cohorts being compared. p<0.0001 when comparing wildtype and mutant cohorts unless otherwise indicated. The bottom line indicated wildtype GAG levels, while the top line indicated GAG levels in the vehicle-treated Idua-W402X mice. p<0.0001 when comparing WT versus W402X cohorts unless otherwise indicated.

FIGS. 9A to 90 show the level of specific GAGs in 10-week-old mouse urine as determined by mass spectrometry. The level of dermatan sulfate (FIG. 9A), heparan sulfate (FIG. 9B), and chondroitin sulfate (FIG. 9C) were determine in mouse urine samples by mouse spectrometry. 120 mg/kg triamterene was administered to 8-week-old Idua-W402X mice via oral gavage for 2 weeks. Urine samples were collected from Idua-W402X mice at the end of treatment as well as from age-matched wild-type and Idua-W402X control mice. The data are shown as the mean+/−SD for each cohort, with each symbol representing the value from an individual mouse. Exact p values calculated from the t-test are shown above the brackets, which indicate the cohorts being compared. P<0.0001 when comparing wild-type and mutant cohorts unless otherwise indicated. No significant differences in chondroitin sulfate were found among the different cohorts.

### DETAILED DESCRIPTION

The disclosed subject matter can be understood more readily by reference to the following detailed description, the Figures, and the examples included herein.

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed. As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

It is understood that the disclosed methods and systems are not limited to the particular methodology, protocols, and systems described as these may vary. It is also to be understood that the terminology used herein is for the purpose of describing particular embodiments only, and is not intended to limit the scope of the present invention which will be limited only by the appended claims.

### Definitions

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

As used in the specification and the appended claims, the singular forms “a,” “an” and “the” include plural referents unless the context clearly dictates otherwise.

The word “or” as used herein means any one member of a particular list and can also include any combination of members of that list.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance can or can not occur, and that the description includes instances where said event or circumstance occurs and instances where it does not.

As used herein, the term “subject” refers to the target of administration, e.g., an animal. Thus, the subject of the herein disclosed methods can be a vertebrate, such as a mammal. The subject of the herein disclosed methods can be a human. The term does not denote a particular age or sex. Thus, adult and newborn subjects, as well as fetuses, whether male or female, are intended to be covered. In one aspect, the subject is a patient. A patient refers to a subject afflicted with a disease or disorder. The term “patient” includes human and veterinary subjects.

The terms “treating”, “treatment”, “therapy”, and “therapeutic treatment” as used herein refer to curative therapy, prophylactic therapy, or preventative therapy. As used herein, the terms refer to the medical management of a subject or a patient with the intent to cure, ameliorate, stabilize, or prevent a disease, pathological condition, or disorder, such as, for example, cancer or a tumor. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder: preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease.

As used herein, the terms “administering” and “administration” refer to any method of providing a composition to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed composition or peptide or pharmaceutical preparation and a cell, target receptor, or other biological entity together in such a manner that the compound can affect the activity of the target (e.g., receptor, transcription factor, cell, etc.), either directly: i.e., by interacting with the target itself, or indirectly; i.e., by interacting with another molecule, co-factor, factor, or protein on which the activity of the target is dependent.

The term “pharmaceutically acceptable” describes a material that is not biologically or otherwise undesirable, i.e., without causing an unacceptable level of undesirable biological effects or interacting in a deleterious manner. As used herein, the term “pharmaceutically acceptable carrier” refers to sterile aqueous or non-aqueous solutions, dispersions, suspensions or emulsions, as well as sterile powders for reconstitution into sterile injectable solutions or dispersions just prior to use. Examples of suitable aqueous and non-aqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

The phrase “genetic disorder”, as used herein, refers to a chronic disorder which is caused by one or more defective genes that are often inherited from the parents, and which can occur unexpectedly when two healthy carriers of a defective recessive gene reproduce, or when the defective gene is dominant. Genetic disorders can occur in different inheritance patterns which include the autosomal dominant pattern wherein only one mutated copy of the gene is needed for an offspring to be affected, and the autosomal recessive pattern wherein two copies of the gene must be mutated for an offspring to be affected. The phrase “genetic disorder”, as used herein, encompasses a genetic disorder, genetic disease, genetic condition or genetic syndrome.

As used herein, a “nonsense mutation” is a point mutation changing a codon corresponding to an amino acid to a stop codon.

As used herein, the terms “effective amount” and “amount effective” refer to an amount that is sufficient to achieve the desired result or to have an effect on an undesired condition.

As used herein, a “therapeutically effective amount” refers to that amount of the compound of the invention or other active ingredient sufficient to provide a therapeutic benefit in the treatment or management of the disease or to delay or minimize symptoms associated with the disease. Further, a therapeutically effective amount with respect to a compound of the invention means that amount of therapeutic agent alone, or in combination with other therapies, that provides a therapeutic benefit in the treatment or management of the disease, Used in connection with an amount of a compound of the invention, the term can encompass an amount that improves overall therapy, reduces or avoids symptoms or causes of disease, or enhances the therapeutic efficacy of or synergies with another therapeutic agent.

As used herein, a “prophylactically effective amount” refers to that amount of a compound of the invention or other active ingredient sufficient to result in the prevention, recurrence or spread of the disease. A prophylactically effective amount may refer to the amount sufficient to prevent initial disease or the recurrence or spread of the disease or the occurrence of the disease in a patient, including but not limited to those predisposed to the disease. A prophylactically effective amount may also refer to the amount that provides a prophylactic benefit in the prevention of the disease. Further, a prophylactically effective amount with respect to a compound of the invention means that amount alone, or in combination with other agents, that provides a prophylactic benefit in the prevention of the disease. Used in connection with an amount of a compound of the invention, the term can encompass an amount that improves overall prophylaxis or enhances the prophylactic efficacy of or synergies with another prophylactic agent.

Triamterene for Suppressing Nonsense Mutations

Disclosed herein are methods of treating or preventing a disease caused by nonsense mutations, or ameliorating one or more symptoms associated therewith, that involve administering to a patient in need thereof a therapeutically or prophylactically effective amount of triamterene, or a pharmaceutically acceptable salt, hydrate, solvate, clathrate, prodrug, or polymorph thereof.

Triamterene, also known as 2,4,7-triamino-6-phenylpteridine, is a well-known pteridine diuretic having the structural formula:

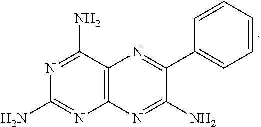 [SMILES: Nc1nc(N)c2nc(-c3ccccc3)c(N)nc2n1]

The diuretic, hypotensive and potassium-saving properties of triamterene, when used alone or in combination with other diuretics, has been known for some time. See, for example, U.S. Pat. No. 3,081,230, hereby incorporated by reference herein in its entirety. Triamterene is currently marketed as DYRENIUM®, in which it is the sole active ingredient, and in a combination with hydrochlorothiazide available under the name DYAZIDE®. The drug is widely used, particularly for its ability to restrict the loss of potassium caused by other diuretics. Gelatin capsule dosage forms of triamterene are disclosed in U.S. Pat. No. 4,255,413, hereby incorporated by reference herein in its entirety. Aldehyde adducts of triamterene are disclosed in U.S. Pat. No. 4,285,947, hereby incorporated by reference herein in its entirety. In some cases, the composition does not comprise hydrochlorothiazide.

In some embodiments, the triamterene is present in a capsule in an amount from about 5 mg to about 100 mg. and the hydrochlorothiazide of from about 2 mg to about 250 mg and long acting propranolol hydrochloride from about 60 mg to 320 mg.

In some embodiments, the subject is a mammal, more preferably a human susceptible to or at risk of acquiring a genetic disease. In some embodiments, the subject has undergone a screening process to determine the presence of a nonsense mutation comprising the steps of screening a subject or cells extracted therefrom by an acceptable nonsense mutation screening assay.

In some embodiments, the subject is an infant or child. In some embodiments, the subject is a fetus. In these embodiments, the method can involve administering triamterene either directly to the fetus or instead to the mother.

Disclosed herein is a method for treating a genetic disorder with a premature stop-codon truncation mutation and/or a protein truncation phenotype, the method comprising administering to a subject in need thereof a therapeutically effective amount of triamterene. Also disclosed herein is triamterene for use in the treatment of a genetic disorder with a premature stop-codon truncation mutation and/or a protein truncation phenotype. Also disclosed herein is triamterene in the manufacture of a medicament for treating a genetic disorder with a premature stop-codon truncation mutation and/or a protein truncation phenotype.

In some embodiments, the genetic disorder is selected from the group consisting of cystic fibrosis (CF), Duchenne muscular dystrophy (DMD), ataxia-telangiectasia, Hurler syndrome and other Mucopolysaccharidoses, hemophilia A, hemophilia B, Usher syndrome, Tay-Sachs, Becker muscular dystrophy (BMD), Congenital muscular dystrophy (CMD), Factor VII deficiency, Familial atrial fibrillation, Hailey-Hailey disease, McArdle disease, Nephropathic cystinosis, Polycystic kidney disease, Rett syndrome, Spinal muscular atrophy (SMA), cystinosis, Severe epidermolysis bullosa, Dravet syndrome, X-linked nephrogenic diabetes insipidus (XNDI), X-linked retinitis pigmentosa, and cancer.

Also disclosed is a method of increasing the expression level of a gene having a premature stop-codon mutation, the method comprising translating the gene into a protein in the presence of triamterene. Also disclosed is triamterene for use in increasing the expression level of a gene having a premature stop-codon mutation. Also disclosed is the use of triamterene in the manufacture of a medicament for increasing the expression level of a gene having a premature stop-codon mutation.

Nonsense Mutations

In genetics, nonsense mutation is a point mutation in a sequence of DNA that results in a premature stop codon, or a nonsense codon in the transcribed mRNA, and in a truncated, incomplete, and usually nonfunctional protein product. The functional effect of a nonsense mutation depends on the location of the stop codon within the coding DNA. For example, the effect of a nonsense mutation depends on the proximity of the nonsense mutation to the original stop codon, and the degree to which functional subdomains of the protein are affected. A nonsense mutation differs from a missense mutation, which is a point mutation where a single nucleotide is changed to cause substitution of a different amino acid.

Despite an expected tendency for premature termination codons to yield shortened polypeptide products, in fact the formation of truncated proteins does not occur often in vivo. Many organisms—including humans and lower species, such as yeast—employ a nonsense-mediated mRNA decay pathway, which degrades mRNAs containing nonsense mutations before they are translated into nonfunctional polypeptides.

Nonsense mutations can cause a genetic disease by preventing complete transcription of a specific protein, for example, dystrophin in Duchenne muscular dystrophy. The same disease may, however, be caused by other kinds of damage to the same gene. Examples of diseases in which nonsense mutations are known to be among the causes include Cystic fibrosis, Duchenne muscular dystrophy, Beta thalassaemia, and Hurler syndrome.

Hurler Syndrome

Hurler syndrome is also known as mucopolysaccharidosis type IH (MPS IH), Hurler's disease, and formerly gargoylism. It is a genetic disorder that results in the buildup of glycosaminoglycans (AKA GAGs, or mucopolysaccharides) due to a deficiency of alpha-L iduronidase, an enzyme responsible for the degradation of GAGs in lysosomes. Without this enzyme, a buildup of dermatan sulfate and heparan sulfate occurs in the body. Symptoms appear during childhood and early death can occur due to organ damage. Hurler syndrome is classified as a lysosomal storage disease. It is clinically related to Hunter syndrome (MPS II); however, Hunter syndrome is X-linked, while Hurler syndrome is autosomal recessive.

Children with Hurler Syndrome carry two defective copies of the IDUA gene, which has been mapped to the 4p16.3 site on chromosome 4. This is the gene which encodes for the protein iduronidase. As of 2001, 52 different mutations in the IDUA gene have been shown to cause Hurler syndrome. Because Hurler syndrome is an autosomal recessive disorder, affected persons have two nonworking copies of the gene.

Diagnosis often can be made through clinical examination and urine tests (excess mucopolysaccharides are excreted in the urine). Enzyme assays (testing a variety of cells or body fluids in culture for enzyme deficiency) are also used to provide definitive diagnosis of one of the mucopolysaccharidoses. Prenatal diagnosis using amniocentesis and chorionic villus sampling can verify if a fetus either carries a copy of the defective gene or is affected with the disorder. Genetic counseling can help parents who have a family history of the mucopolysaccharidoses determine if they are carrying the mutated gene that causes the disorders.

Cystic Fibrosis

Cystic fibrosis (CF) is a genetic disorder that affects mostly the lungs, but also the pancreas, liver, kidneys, and intestine. Long-term issues include difficulty breathing and coughing up mucus as a result of frequent lung infections. Other signs and symptoms may include sinus infections, poor growth, fatty stool, clubbing of the fingers and toes, and infertility in most males. Different people may have different degrees of symptoms.

CF is caused by a mutation in the gene cystic fibrosis transmembrane conductance regulator (CFTR). Although most people have two working copies (alleles) of the CFTR gene, only one is needed to prevent cystic fibrosis. CF develops when neither allele can produce a functional CFTR protein. Thus, CF is considered an autosomal recessive disease. Several mutations in the CFTR gene can occur, and different mutations cause different defects in the CFTR protein, sometimes causing a milder or more severe disease. These protein defects are also targets for drugs which can sometimes restore their function. ΔF508-CFTR, which occurs in >90% of patients in the U.S., creates a protein that does not fold normally and is not appropriately transported to the cell membrane, resulting in its degradation. Nonsense mutations result in proteins that are too short (truncated) because production is ended prematurely. Other mutations produce proteins that do not use energy (in the form of ATP) normally, do not allow chloride, iodide, and thiocyanate to cross the membrane appropriately, and degrade at a faster rate than normal. Mutations may also lead to fewer copies of the CFTR protein being produced.

Cystic fibrosis may be diagnosed by many different methods, including newborn screening, sweat testing, and genetic testing. As of 2006 in the United States, 10% of cases are diagnosed shortly after birth as part of newborn screening programs. The newborn screen initially measures for raised blood concentration of immunoreactive trypsinogen. Infants with an abnormal newborn screen need a sweat test to confirm the CF diagnosis. In many cases, a parent makes the diagnosis because the infant tastes salty, Immunoreactive trypsinogen levels can be increased in individuals who have a single mutated copy of the CFTR gene (carriers) or, in rare instances, in individuals with two normal copies of the CFTR gene. The most commonly used form of testing is the sweat test. Sweat testing involves application of a medication that stimulates sweating (pilocarpine). To deliver the medication through the skin, iontophoresis is used, whereby one electrode is placed onto the applied medication and an electric current is passed to a separate electrode on the skin. The resultant sweat is then collected on filter paper or in a capillary tube and analyzed for abnormal amounts of sodium and chloride. People with CF have increased amounts of them in their sweat. In contrast, people with CF have less thiocyanate and hypothiocyanite in their saliva and mucus. In the case of milder forms of CF, transepithelial potential difference measurements can be helpful. CF can also be diagnosed by identification of mutations in the CFTR gene.

Women who are pregnant or couples planning a pregnancy can have themselves tested for the CFTR gene mutations to determine the risk that their child will be born with CF. Testing is typically performed first on one or both parents and, if the risk of CF is high, testing on the fetus is performed.

Duchenne Muscular Dystrophy

Duchenne muscular dystrophy (DMD) is a severe type of muscular dystrophy. The symptom of muscle weakness usually begins around the age of four in boys and worsens quickly. Typically muscle loss occurs first in the upper legs and pelvis followed by those of the upper arms. This can result in trouble standing up. Most are unable to walk by the age of 12. Affected muscles may look larger due to increased fat content. Scoliosis is also common. Some may have intellectual disability. Females with a single copy of the defective gene may show mild symptoms.

The disorder is X-linked recessive. About two thirds of cases are inherited from a person's parents, while one third of cases are due to a new mutation. It is caused by a mutation in the gene for the protein dystrophin. Dystrophin is important to maintain the muscle fiber's cell membrane. Genetic testing can often make the diagnosis at birth. Those affected also have a high level of creatine kinase in their blood.

DMD affects about one in 5,000 males at birth. It is the most common type of muscular dystrophy. The average life expectancy is 26; however, with excellent care, some may live into their 30s or 40s, Gene therapy, as a treatment, is in the early stages of study in humans.

DMD is caused by a mutation of the dystrophin gene at locus Xp21, located on the short arm of the X chromosome. Dystrophin is responsible for connecting the cytoskeleton of each muscle fiber to the underlying basal lamina (extracellular matrix), through a protein complex containing many subunits. The absence of dystrophin permits excess calcium to penetrate the sarcolemma (the cell membrane). Alterations in calcium and signalling pathways cause water to enter into the mitochondria, which then burst.

Genetic counseling is advised for people with a family history of the disorder. DMD can be detected with about 95% accuracy by genetic studies performed during pregnancy.

Beta Thalassemias

Beta thalassemias are a group of inherited blood disorders caused by reduced or absent synthesis of the beta chains of hemoglobin that result in variable outcomes ranging from severe anemia to clinically asymptomatic individuals. Global annual incidence is estimated at one in 100,000. Beta thalassemias are caused by mutations in the HBB gene on chromosome 11, inherited in an autosomal recessive fashion. The severity of the disease depends on the nature of the mutation.

HBB blockage over time leads to decreased beta-chain synthesis. The body's inability to construct new beta-chains leads to the underproduction of HbA. Reductions in HbA available overall to fill the red blood cells in turn leads to microcytic anemia. Microcytic anemia ultimately develops in respect to inadequate HBB protein for sufficient red blood cell functioning. Due to this factor, the patient may require blood transfusions to make up for the blockage in the beta-chains. Repeated blood transfusions can lead to build-up of iron overload, ultimately resulting in iron toxicity. This iron toxicity can cause various problems, including myocardial siderosis and heart failure leading to the patient's death.

Two major groups of mutations can be distinguished: non-deletion forms and a deletion form. Non-deletion forms, in general, involve a single base substitution or small insertions near or upstream of the β globin gene. Most often, mutations occur in the promoter regions preceding the beta-globin genes. Less often, abnormal splice variants are believed to contribute to the disease. Deletion forms involve deletions of different sizes involving the β globin gene produce different syndromes such as (βo) or hereditary persistence of fetal hemoglobin syndromes.

All beta thalassemias may exhibit abnormal red blood cells, a family history is followed by DNA analysis. This test is used to investigate deletions and mutations in the alpha- and beta-globin-producing genes. Family studies can be done to evaluate carrier status and the types of mutations present in other family members. DNA testing is not routine, but can help diagnose thalassemia and determine carrier status. In most cases the treating physician uses a clinical pre-diagnosis assessing anemia symptoms: fatigue, breathlessness and poor exercise tolerance. Further genetic analysis may include HPLC should routine electrophoresis prove difficult.

Affected children require regular lifelong blood transfusion and can have complications, which may involve the spleen.

Patient Selection

The disclosed compositions and methods can be used in some embodiments to treat any subject that has been diagnosed with a disease or condition caused by a nonsense mutation. In some embodiments, the disclosed methods further involve assaying a sample from the subject for the presence of a nonsense mutation known or determined to cause a disease, and treating the subject with triamterene if a nonsense mutation is detected.

Therefore, in some embodiments, it is first determined that the patient is suffering from a disease associate with premature translation termination and/or nonsense-mediated mRNA decay. In some embodiments, the subject has undergone a screening process to determine the presence of a nonsense mutation comprising the steps of screening a subject, or cells extracted therefrom, by an acceptable nonsense mutation screening assay. In some embodiments, the DNA of the subject can be sequenced or subject to Southern Blot, polymerase chain reaction (PCR), use of the Short Tandem Repeat (STR), or polymorphic length restriction fragments (RFLP) analysis to determine if a nonsense mutation is present in the DNA of the patient. Alternatively, it can be determined if altered levels of the protein with the nonsense mutation are expressed in the patient by western blot or other immunoassays. In another embodiment, the subject is an unborn child who has undergone screening in utero for the presence of a nonsense mutation. Administration of triamterene can occur either before or after birth.

In some embodiments, cells from the subject are screened for premature translation termination and/or nonsense-mediated mRNA decay with a method such as that described above (i.e., the DNA of the cell can be sequenced or subjected to Southern Blot, polymerase chain reaction (PCR), use of the Short Tandem Repeat (STR), or polymorphic length restriction fragments (RFLP) analysis to determine if a nonsense mutation is present in the DNA of the cell).

In certain embodiments, triamterene can be used in combination with at least one other therapeutic agent. Therapeutic agents include, but are not limited to non-opioid analgesics; non-steroid anti-inflammatory agents; antiemetics; β-adrenergic blockers; anticonvulsants; antidepressants; Ca2+-channel blockers; anticancer agent and mixtures thereof.

In certain embodiments, triamterene can be administered or formulated in combination with anticancer agents. Suitable anticancer agents include, but are not limited to, alkylating agents; nitrogen mustards; folate antagonists; purine antagonists; pyrimidine antagoinists; spindle poisons; topoisomerase inhibitors; apoptosis inducing agents; angiogenesis inhibitors; podophyllotoxins; nitrosoureas; cisplatin; carboplatin; interferon; asparginase; tamoxifen; leuprolide; flutamide; megestrol; mitomycin; bleomycin; doxorubicin; irinotecan and taxol.

In certain embodiments, triamterene can be administered or formulated in combination with antibiotics. In certain embodiments, the antibiotic is a macrolide (e.g., tobramycin (Tobi®)), a cephalosporin (e.g., cephalexin (Keflex®), cephradine (Velosef®), cefuroxime (Ceftin®), cefprozil (Cefzil®), cefaclor (Ceclor®), cefixime (Suprax®) or cefadroxil (Duricef®)), a clarithromycin (e.g., clarithromycin (Biaxin®)), an erythromycin (e.g., erythromycin (EMycin®)), a penicillin (e.g. penicillin V (V-Cillin K® or Pen Vee K®)) or a quinolone (e.g. ofloxacin (Floxin®), ciprofloxacin (Cipro®) or norfloxacin (Noroxin®)). In a preferred embodiment, the antibiotic is active against Pseudomonas aeruginosa.

In some cases, triamterene and the other therapeutics agent can act additively or, more preferably, synergistically. In some embodiments, a composition comprising triamterene is administered concurrently with the administration of another therapeutic agent, which can be part of the same composition or in a different composition from that comprising triamterene. In another embodiment, triamterene is administered prior to or subsequent to administration of another therapeutic agent.

The magnitude of a prophylactic or therapeutic dose of a particular active ingredient in the acute or chronic management of a disease or condition will vary, however, with the nature and severity of the disease or condition, and the route by which the active ingredient is administered. The dose, and perhaps the dose frequency, will also vary according to the age, body weight, and response of the individual patient. Suitable dosing regimens can be readily selected by those skilled in the art with due consideration of such factors. In general, the recommended daily dose range for the conditions described herein lie within the range of from about 0.1 mg to about 2000 mg per day. In one embodiment, triamterene is given as a single once-a-day dose. In another embodiment, triamterene is given as divided doses throughout a day. More specifically, the daily dose is administered in a single dose or in equally divided doses. In some cases, a daily dose range should be from about 5 mg to about 500 mg per day, such as, between about 10 mg and about 200 mg per day. It may be necessary to use dosages of triamterene outside the ranges disclosed herein in some cases, as will be apparent to those of ordinary skill in the art. Furthermore, it is noted that the clinician or treating physician will know how and when to interrupt, adjust, or terminate therapy in conjunction with individual patient response.

Pharmaceutical Compositions

Pharmaceutical compositions and single unit dosage forms comprising triamterene, or a pharmaceutically acceptable polymorph, prodrug, salt, solvate, hydrate, or clathrate thereof, are contemplated for used in the disclosed methods. Individual dosage forms may be suitable for oral, mucosal (including sublingual, buccal, rectal, nasal, or vaginal), parenteral (including subcutaneous, intramuscular, bolus injection, intraarterial, or intravenous), transdermal, or topical administration. Pharmaceutical compositions and dosage forms typically also comprise one or more pharmaceutically acceptable excipients.

Single unit dosage forms are suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g. aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms will typically vary depending on their use. For example, a dosage form used in the acute treatment of inflammation or a related disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease or disorder. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

Typical pharmaceutical compositions and dosage forms comprise one or more carriers, excipients or diluents. Suitable excipients are well known to those skilled in the art of pharmacy, and non-limiting examples of suitable excipients are provided herein, Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form.

Also contemplated are anhydrous pharmaceutical compositions and dosage forms, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp, 379-80. In effect, water and heat accelerate the decomposition of some compounds, Thus, the effect of water on a formulation can be of great significance since moisture and/or humidity are commonly encountered during manufacture, handling, packaging, storage, shipment, and use of formulations. Anhydrous pharmaceutical compositions and dosage forms can be prepared using anhydrous or low moisture containing ingredients and low moisture or low humidity conditions.

Also contemplated are pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

A number of embodiments of the invention have been described, Nevertheless, it will be understood that various modifications may be made without departing from the spirit and scope of the invention. Accordingly, other embodiments are within the scope of the following claims.

### EXAMPLES

### Example 1

Introduction

A nonsense mutation is a gene lesion that introduces an in-frame premature termination codon (FTC) within the open reading frame of an mRNA. When a FTC-containing mRNA undergoes translation, the FTC triggers two mechanisms that reduce the amount of functional protein generated from the mRNA. First, a FTC terminates translation before a full-length protein can be made, resulting in a truncated polypeptide that lacks normal function or is unstable. Second, a FTC can activate nonsense-mediated mRNA decay (NMD), a conserved eukaryotic pathway that targets FTC-containing mRNAs for degradation, NMD prevents FTC-containing mRNAs from undergoing subsequent rounds of translation, further reducing the amount of functional protein that can be generated. Significantly, 11% of all mutations associated with genetic diseases generate an in-frame FTC (Mort, M., et al, (2008) Hum. Mutat. 29:1037-1047), indicating that a significant number of patients harbor a PTC.

One way to overcome the effects of nonsense mutations is to suppress translation termination at PTCs to restore partial expression of full-length, functional protein. A number of low molecular weight compounds have been identified that suppress translation termination at a FTC through a mechanism termed as “readthrough” (Lee, H. L., et al. (2012) Pharmacol. Ther. 136:227-266). Readthrough occurs when an aminoacyl-tRNA that can base pair with two of the three nucleotides of a stop codon is accommodated into the ribosomal acceptor site and its bound amino acid becomes inserted into the nascent polypeptide at the FTC position (Roy, B., et al, (2016) Proc. Natl. Acad. Sci. U.S.A. 113:12508-12513; Xue, X., et al, (2017) Hum. Mol. Genet). This readthrough mechanism permits translation elongation to continue in the correct ribosomal reading frame to generate a full-length polypeptide.

The use of small molecules to promote readthrough could potentially be developed as a therapy to treat patients who carry a FTC. Currently, the best characterized readthrough-inducing agents include the aminoglycosides, a well-characterized class of antibiotic (Howard, M., et al. (1996) Nat. Med. 2:467-469; Bedwell, D. M., et al. (1997) Nat. Med. 3:1280-1284), and ataluren, a non-antibiotic compound (Welch, E. M., et al. (2007) Nature 447:87-91). While some aminoglycosides have been shown to be effective at suppressing PTCs in vitro and in vivo (Lee, H. L., et al. (2012) Pharmacol. Ther, 136:227-266; Keeling, K. M., et al. (2014) Annu Rev Genomics Hum Genet. 15:371-394), aminoglycosides are precluded from long-term clinical administration due to their potential to cause ototoxicity (Huth, M. E., et al. (2011) International Journal of Otolaryngology 2011:1-19) and nephrotoxicity (Lopez-Novoa, J. M., et al. (2011) Kidney Int. 79:33-45). Through recent medicinal chemistry efforts, so-called “designer” aminoglycosides have been generated that are more effective than traditional aminoglycosides at suppressing PTCs in mammalian cells but significantly less toxic (Nudelman, I., et al. (2006) Bioorg. Med. Chem. Lett. 16:6310-6315; Hainrichson, M., et al. (2008) Org Biomol Chem 6:227-239; Pokrovskaya, V., et al. (2010) Methods Enzymol. 478:437-462; Kandasamy, J., et al. (2012) J. Med. Chem. 55:10630-10643; Shulman, E., et al. (2014) J. Biol. Chem. 289:2318-2330; Nagel-Wolfrum, K., et al. (2016) BioDrugs 30:49-74). However, these designer aminoglycosides have not yet been shown to be safe for long-term clinical use. Conversely, ataluren, which was identified in a high throughput screen to identify new readthrough agents, has been shown to be non-toxic and safe for clinical use (Hirawat, S., et al. (2004) Pediatr. Pulmonol. 38:248; Hirawat, S., et al. (2007) J. Clin. Pharmacol. 47:430-444). However, recent clinical trials showed that ataluren did not significantly improve lung function in cystic fibrosis patients who carry a PTC (Kerem, E., et al. (2014) Lancet Respir Med. 2:539-547), suggesting that it was not an efficacious enough readthrough agent to alleviate the disease. Additional studies will be needed to determine whether ataluren is effective at alleviating other disorders caused by PTCs. Overall, these data suggest the need for additional safe and effective nonsense suppression compounds.

One way to potentially improve the efficacy of readthrough is to inhibit NMD. By increasing the levels of PTC-containing mRNAs, greater levels of full-length protein can be restored via readthrough. Because a subset of the same translational factors functions in both translation termination and NMD (Kashima, I., et al. (2006) Genes Dev. 20:355-367), it is likely that some compounds may be found that can affect both pathways simultaneously. For example, the anti-inflammatory compound, amlexanox, has been shown to not only mediate PTC readthrough at low levels, but to also weakly attenuate NMD (Gonzalez-Hilarion, S., et al. (2012) Orphanet Journal of Rare Diseases 7:58). More effective compounds can be identified that possess the ability of promoting readthrough while also inhibiting NMD. Furthermore, dual function compounds are likely to restore more full-length, functional protein than compounds that induce readthrough or inhibit NMD alone.

In the current study, NanoLuc reporters were used to screen a library of 10,000 low molecular weight compounds for those that induce readthrough, inhibit NMD, or both. The goal of this screen was to identify compounds that suppress the Idua-W402X nonsense mutation that is associated with the lysosomal storage disease, Mucopolysaccharidosis I-Hurler (MPS I-H). MPS 1-H is caused by a severe deficiency of the lysosomal enzyme, α-L-iduronidase, which participates in the catabolism of the glycosaminoglycans (GAGs), dermatan sulfate and heparan sulfate. Loss of α-L-iduronidase results in the progressive accumulation of GAGs, leading to the onset of abnormalities in the joints, bone, heart, liver, lung and neurological tissues, and a reduced lifespan.

Triamterene and doxazocin, two FDA-approved anti-hypertension drugs, were identified from the screen as FTC suppression compounds that appear to readthrough the Idua-W402X mutation in a mouse model of MPS I-H. Both compounds restored α-L-iduronidase activity in mouse embryonic fibroblasts (MEFs) homozygous for the Idua-W402X mutation in a dose-dependent manner and correspondingly, significantly reduced GAG accumulation. The ability of triamterene, but not doxazocin, to suppress the Idua-W402X mutation could be independently confirmed in HEK293 cells expressing a dual luciferase readthrough reporter. Examination of Idua steady state mRNA levels showed that triamterene did not alter Idua mRNA levels in wildtype MEFs, but significantly increased Idua mRNA levels in Idua-W402X MEFs, suggesting that triamterene increases the stability of the Idua-W402X mRNA, potentially through NMD inhibition. Comparison of the α-L-iduronidase activity and GAG levels elicited by triamterene in Idua-W402X MEFs versus MEFs carrying an insertional mutation in the Idua locus that is not amenable to readthrough showed that triamterene restores α-L-iduronidase primarily through a FTC-dependent mechanism. Because triamterene treats hypertension by inhibiting epithelial sodium channels (ENaC) in the distal kidney, we tested whether another ENaC inhibitor, amiloride (Bubien, J. K. (2010) J. Biol. Chem. 285:23527-23531), also affected α-L-iduronidase and GAG levels. In contrast to triamterene, amiloride did not increase α-L-iduronidase or decrease GAG levels in Idua-W402X MEFs. Finally, it was examined whether in vivo triamterene administration to Idua-W402X mice altered GAG accumulation. A dose-dependent decrease in GAG accumulation was observed in bone, brain, heart, kidney, liver, lung and spleen, with GAG levels completely normalized in all tissues when mice were administered 120 mg/kg triamterene. Consistent with a reduction of GAG accumulation in the mouse tissues, there was a significant reduction in dermatan sulfate and heparan sulfate in the urine of Idua-W402X mice similarly dosed with triamterene. Together, these data suggest that triamterene suppresses the Idua-W402X mutation and represents a promising new drug for treating MPS I-H in patients who carry this nonsense mutation.

Materials and Methods

Tissue Culture:

Mouse embryonic fibroblasts were immortalized using the SV40 large T antigen. MEFs utilized in this study included MEFs derived from homozygous: wildtype mice (WT), knock-in MPS I-H mice carrying a genomic point mutation that generates a PTC that is homologous to the IDUA W402X nonsense mutation (Idua-W402X), and knock-out MPS I-H mice carrying an insertional mutation in the mouse Idua locus (Idua-KO). MEF cell lines were cultured at 37° C. with 6.5% CO₂ in Dulbecco's Modification of Eagle's Medium containing 4.5 g/L glucose, L-glutamine and sodium pyruvate (Corning Cellgro 10-013-CV). This media was supplemented with 100 units/rd penicillin/streptomycin (Corning Cellgro 30-002-CI), MEM non-essential amino acids (Corning Cellgro 25-025-CI) at a final concentration of 1% (v/v), and fetal bovine sera (Atlanta Biologicals S11150) at a final concentration of 10% (v/v). HEK293 cells were cultured similarly. Fisher rat thyroid (FRT) cells were cultured in Nutrient Mixture F-12 Coon's modification media (Sigma F6636) supplemented with 5% fetal bovine sera. For stably transfected HEK293 and FRT cells, 100 units/ml penicillin/streptomycin was added to the media in the absence of zeocin to prevent bacterial contamination.

Construction of the Readthrough Reporters:

The NanoLuc RT/NMD reporter was constructed using the pFN[Nluc/CMV/neo] plasmid (Promega CS181701), which contained the NanoLuc open reading frame. Initially, a multi-cloning site was placed into the XhoI/NotI sites of this plasmid using the annealed oligos DB4078 5′-TCGAGCCAAG CTTGCATGCCT GCAGGTCGACT CTAGAGGATCC CCGGGGAATTCGC-3′ (SEQ ID NO:1) and DB4079 5′-GGCCGCGAAT TCCCCGGGGA TCCTCTAGAG TCGACCTGCAG GCATGCAAGCTTGGC-3′ (SEQ ID NO:2) to remove the barnase sequence for subsequent cloning. This new construct (pDB1333) was used as a template to generate the W134X (UGA) premature termination codon in the NanoLuc gene (pDB1345) using site directed mutagenesis with the forward primer DB4144, 5′-gggaccctgt gaaacggcaac-3′ (SEQ ID NO:3) and the reverse primer DB4145, 5′-gttgccgttt cacagggtccc-3′ (SEQ ID NO:4). The final W134X NanoLuc RT/NMD reporter was generated by replacing the Renilla gene within the Renilla-β-globin/pcDNA3.1Zeo(−) plasmid (pDB1329) with the W134X Nanoluc gene from pDB1345 using NheI/XhoI sites such that the NanoLuc gene was fused in-frame with exon 1 of 3-globin.

The construction of the Idua-W402X dual luciferase reporters has been previously described (Wang, D., et al. (2012) Mol. Genet. Metab, 105:116-125). The p2luc construct was modified to express either the WT mouse Idua codon (UGG) that is homologous to W402 codon in the human IDUA cDNA, or the Idua W402X premature stop codon (UAG), along with three codons of upstream and downstream mouse Idua sequence. Complementary oligonucleotides for generating the Idua-W402X construct: 5′-TCGACGGAACAACTCTAGGCAGAGGTCG-3′ (SEQ ID NO:5); 5′-GATCCGACCTCTGCCTAGAGTTGTTCCG-3′ (SEQ ID NO:6), and the WT Idua construct: 5′-TCGACGGAACAACTCTGGGCAGAGGTCG-3′ (SEQ ID NO:7); 5′-GATCCGACCTCTGCCCAGAGTTGTTCCG-3′ (SEQ ID NO:8) were annealed to generate double-stranded DNA fragments that were ligated into the SalI and BamHI restriction sites of the p2luc vector, yielding the Idua-W402X (pDB1134) and Idua-WT (pDB1133) p2luc constructs. Using the NotI and NheI restriction sites, the Idua-W402X and Idua-WT dual luciferase constructs were subcloned into pcDNA3.1Zeo(+) to generate pDB1325 and pDB1326, respectively, for stable expression in mammalian cells.

Generation of Readthrough Reporter Cells:

The W134X NanoLuc RT/NMD reporter was linearized with BlgII and transfected into FRT cells using Lipofectamine 2000 (Invitrogen 11668). Stably transfected FRT cells were selected by the addition of 800 μg/ml zeocin to the growth media for 2-3 weeks. Stable FRT reporter cell lines were maintained by the addition of 200 μg/ml of zeocin to the media. Monoclonal FRT cell lines were established by collecting cells from single-cell-derived colonies and expanding them.

The Idua-W402X and Idua-WT dual luciferase constructs (pDB1325 and pDB1326, respectively) were linearized with BlgII and transfected into HEK293 cells, which were cultured in the presence of 200 μg/ml of zeocin for 2-3 weeks to select for cells stably expressing the reporters. Reporter HEK293 cells were also maintained in 200 μg/ml of zeocin.

Luciferase Assays:

Equal numbers of the W134X NanoLuc RT/NMD monoclonal reporter cells were seeded into 96-well plates and cultured without zeocin for 24 hours. The cells were then treated with screening compounds for 24-hours. Dimethylsulfoxide served as the vehicle in which compounds were administered at a final concentration of 60 μM or 30 μg/ml. After treatment, the cells were washed once with phosphate-buffered saline, followed by incubation with passive lysis buffer (PLB) (Promega, E1941) for 20 minutes at room temperature. NanLuc activity in cell lysates was assayed with the Nano-Glo Luciferase Assay System (Promega, N1120) using the GloMax® Discover System (Promega). NanoLuc activity was normalized to the lysate protein concentration, which was determined by the Bio-Rad Protein Assay Reagent (Bio-Rad, 500-006).

HEK293 dual luciferase reporter cells were similarly cultured, treated with screening compounds, and lysed as described above. Dual luciferase activity was assayed with the Dual Luciferase Assay System (Promega) using the GloMax Multi Detection System (Promega). The percent readthrough was calculated as the ratio of firefly/Renilla luciferase units expressed from the W402X construct relative to the WT construct x 100.

MEF α-L-iduronidase Activity Assay:

MEFs were seeded into 6-well culture dishes at a density of 1.5×10⁵ cells per well. MEFs were grown to 50% confluency and then treated with screening agents for 48 hours. MEFs were subsequently washed with PBS and lysed in Mammalian Protein Extraction Reagent (Pierce) containing a protease inhibitor cocktail (Roche). The total protein concentration was determined using the Bio-Rad Protein Assay, Approximately 75 micrograms of total lysate protein was incubated in a 70-microliter reaction containing 0.12 mM 4-methyl-umbelliferyl-α-L-iduronide (Gold Biotech) and 0.42 mg/ml of D-saccharic acid 1,4-lactone monohydrate (a β-glucuronidase inhibitor) (Sigma 50375) in 130 mM sodium formate buffer, pH 3.5. The reaction was incubated for 72 hours at 37° C. and then quenched with 300 μl of glycine buffer, pH 10.8. 200 μl of each sample was transferred to a 96-well plate (Corning 3631) and fluorescence was measured at an excitation=365 nm and an emission=450 nm using the GloMax® Discover System (Promega). Free acid 4-methylumbelliferone (FMU) (Sigma M1381) in glycine buffer was used to generate a standard curve. Specific activity was calculated as nanomoles of FMU released per milligram of protein per hour. α-L-iduronidase activity remained linear over the 72-hour incubation time.

MEF Glycosaminoglycan Quantitation:

MEFs were seeded into 6-well culture dishes at a density of 5×10⁴ cells per well. MEFs were grown to 50% confluency and then treated with screening agents for 48 hours. Subsequently, the media was removed and the cells were scraped into 500 μl of 0.2 mg/ml papain in pH 6.5 phosphate buffer containing 0.6 mg/ml cysteine. The cell lysates were incubated in the papain solution at 65° C. with gentle agitation for 3 hours. Samples were briefly microfuged to remove debris. GAG levels were determined using the Blyscan Sulfated GAG Assay (Biocolor Ltd, UK). Briefly, 50 μl of each supernatant was mixed with 500 μl of the Blyscan Dye Reagent to bind sulfated GAGs. The dye-bound GAGs were pelleted by microfuging for 10 minutes at 10,000 g at room temperature. 500 μl of the Blyscan Dye Dissociation Reagent was added to each sample to dissociate the GAGs from the dye. 200 μl of each sample was transferred to a 96-well plate (Corning 3631) and the absorbance was measured at a wavelength of 650 nm absorbance using a GloMax® Discover System (Promega). The total amount of sulfated GAGs precipitated from each sample was determined from a chondroitin 4-sulfate (Sigma C9819) standard curve. The total protein concentration in each lysate was determined using the Bio-Rad Protein Assay from a standard curve generated using bovine serum albumin. The GAG levels are expressed as nanograms of GAGs per milligram of total protein.

Quantitating Steady State Idua mRNA Levels:

Total RNA was isolated from MEFs using the Ambion RiboPure kit and DNase-treated using the Turbo DNA-Free kit. Polyadenylated RNA was reverse transcribed into cDNA in a 50 μl reaction containing 1 mg of total RNA; 0.5 mg/ml oligo dT; 1.2 mM dNTPs; 40 U RNasin (Promega); 10 ml of 5×AMV RT buffer and 40 U AMV reverse transcriptase (Promega), RT reactions were incubated at 42° C. for 1.5 hours, and then heat inactivated at 65° C. for 15 minutes. The cDNA was ethanol precipitated and subjected to qPCR in a 25 μl reaction containing 12.5 μl iQ SYBR Green Supermix (Bio-Rad); 0.2 mM of each forward and reverse primer; and 2 mg of cDNA. The following primers sets (forward=Pf and reverse=Pr) were used: Idua 5′-TGACAATGCCTTCCTGAGCTACCA-3′ (SEQ ID NO:9) and Idua Pr: 5′-TGACTGTGAGTACTGGCTTTCGCA-3′ (SEQ ID NO:10); Gapdh 5′-TTCCAGTATGACTCCACTCACGG-3′ (SEQ ID NO:11) and Gapdh Pr: 5′-TGAAGACACCAGTAGACTCCACGAC-3′ (SEQ ID NO:12); Rpl13a Pf: 5′-ATGACAAGAAAAAGCGGATG-3′ (SEQ ID NO: 13) and Rpl13a Pr: 5′-CTTTTCTGCCTGTTTCCGTA-3′ (SEQ ID NO:14). qPCR was performed using the CFX96 Real-Time PCR Detection System (Bio-Rad) using a program that included an initial 3-minute denaturation step at 95° C. followed by 40 repeated cycles of a 10 second denaturation step at 95° C. and a 30 second annealing/extension step at 55° C.

Melt curve analysis was initially performed with each primer set to verify that only one gene product was generated from the PCR reactions. A standard curve was also performed using each primer set to ensure that under the PCR conditions used, the efficiency ranged between 90-110%. The average quantification cycle (Cq) was determined for each mRNA, and mRNA quantification was performed using the Livak (DDCq) method (Livak K J, et al. (2001) Methods. 25(4):402-8), where Gapdh and Rpl13a served as normalization controls. Cq values among the different samples for the various transcripts ranged from 10-34. qPCR was performed using at least 8-12 replicates for each gene product from each sample.

Animal Treatment:

Triamterene was suspended in a vehicle composed of 0.5% methyl cellulose in an artificially sweetened Kool-Aid solution. This mixture was administered orally to 8- to 10-week-old mice via once daily gavage for a total of 14 days. Doses ranged from 30-120 mg/kg. All animal work was conducted according to relevant national and international guidelines. All animal protocols used in this study were reviewed and approved by the UAB IACUC (protocol IACUC-20364).

GAG Quantitation in Mouse Tissues:

This assay was performed as previously described (Wang, D., et al. (2012) Mol. Genet. Metab. 105:116-125: Gunn, G., et al. (2013) Mol. Genet. Metab. 111:374-381). Tissues were homogenized using a Tissue Tearor homogenizer in chloroform:methanol (2:1 v/v). Defatted tissue was dried in a speedvac and then suspended in 100 mM dibasic sodium phosphate, pH 6.5 containing 0.6 mg/ml cysteine and 2 mg/ml papain (Sigma P4762). The mixture was digested at 60° C. for 18-24 hours with constant agitation. The samples were then microfuged at 10,000 g for 15 minutes and the supernatant was used to quantitate the tissue GAGs using the Blyscan Sulfated GAG Assay (Biocolor Ltd, UK). The total amount of sulfated GAGs precipitated from each sample was determined from a standard curve using chondroitin 4-sulfate (Sigma C9819). The GAG levels are expressed as the micrograms of GAGs per milligram of defatted, dried tissue.

Results

Identification of Triamterene in a High Through Screen for PTC Suppression Compounds.

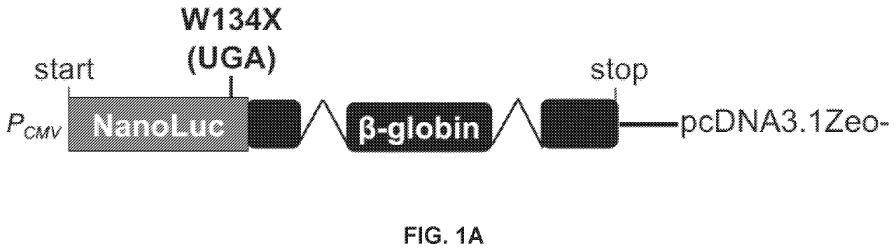

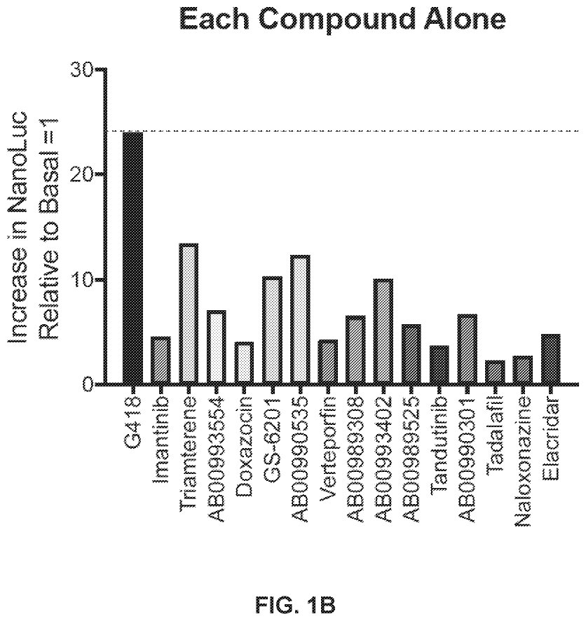

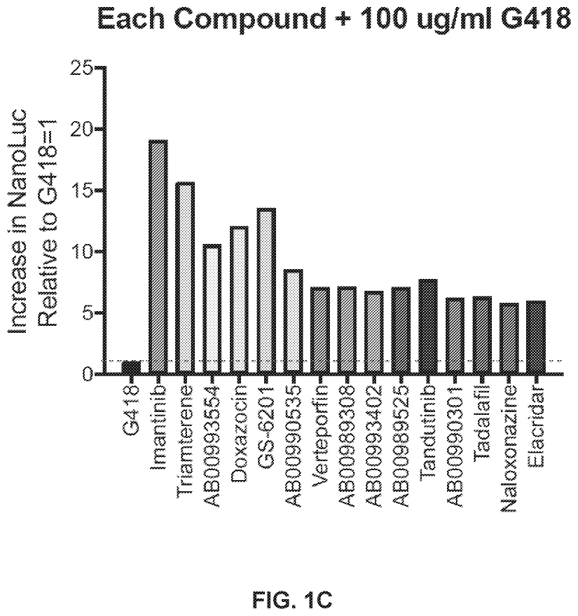

In order to identify new compounds with the ability to suppress PTCs by inducing readthrough, inhibiting NMD, or both, a NanoLuc reporter that harbors a point mutation at codon W134 was utilized, resulting in the introduction of a UGA termination codon (W134X) into the NanoLuc cDNA. This NanoLuc construct was fused in-frame to a human β-globin construct containing intronic regions, which allows the W134X PTC to elicit exon-junction-complex dependent NMD (FIG. 1A). This NanoLuc readthrough/NMD reporter was stably expressed in Fischer rat thyroid cells, which were used to screen a 10,000 low molecular weight compound library for compounds that increase NanoLuc activity in treated cells relative to a vehicle control.

Molecules were recently identified that do not possess readthrough activity themselves, but they can enhance the efficiency of other readthrough compounds. Although the mode of action of one of these “enhancer” molecules, CDX-1, has not been identified, this compound was found to enhance the readthrough mediated by the potent, but toxic, G418 aminoglycoside by 180-fold (Baradaran-Heravi, A., et al. (2016) Nucleic Acids Res, 44:6583-6598), To identify potential readthrough “enhancer” molecules and to amplify the signal of molecules with weak readthrough activity, the NanoLuc reporter screen was performed with each compound alone as well as in combination with 100 μg/ml G418.

The 15 molecules that produced the highest levels of readthrough in combination with G418 are shown in Table 1 and in FIG. 1B-C. While none of the molecules alone were more efficient than G418 at suppressing the NanoLuc W134X PTC (FIG. 10), these compounds significantly amplified the readthrough generated by G418 by 5- to 18-fold (FIG. 1B). These 15 hits could potentially increase NanoLuc reporter activity by acting as weak readthrough agents, NMD inhibitors, readthrough enhancers, as well as by acting through PTC-independent mechanisms (i.e. promoter effects).

Examination of Triamterene for the Ability to Alleviate MPS I-H Biochemical Endpoints in Idua-W402X MEFs.

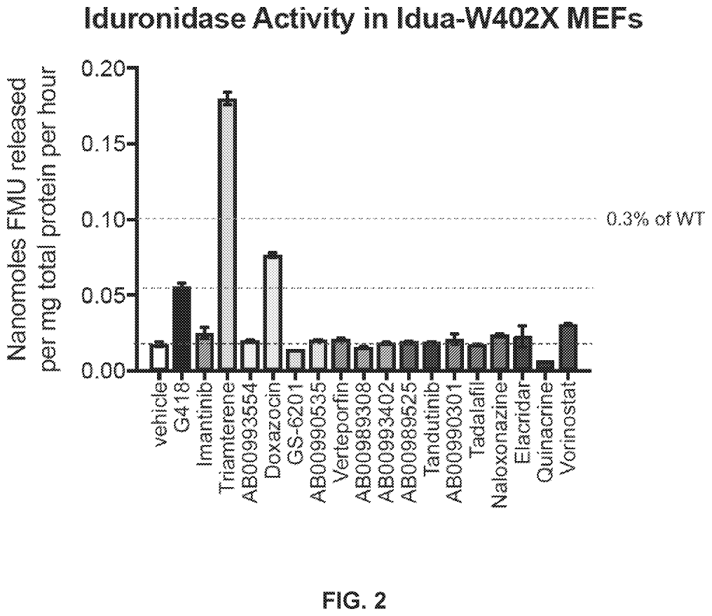

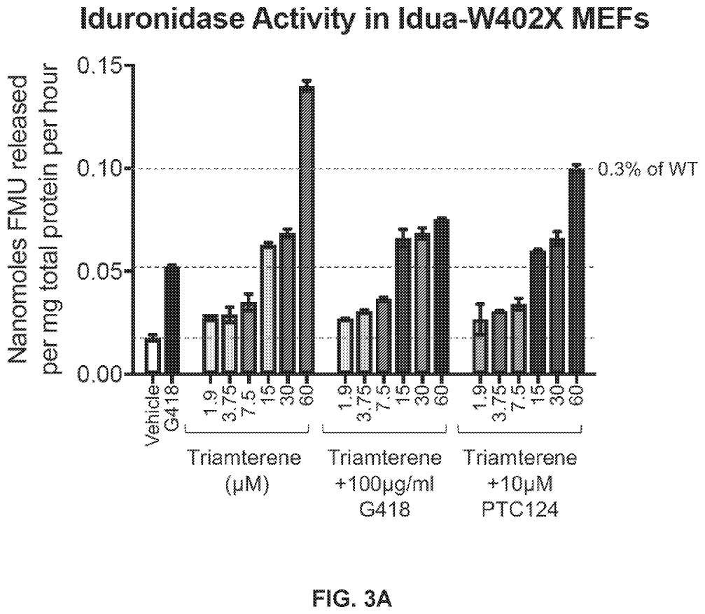

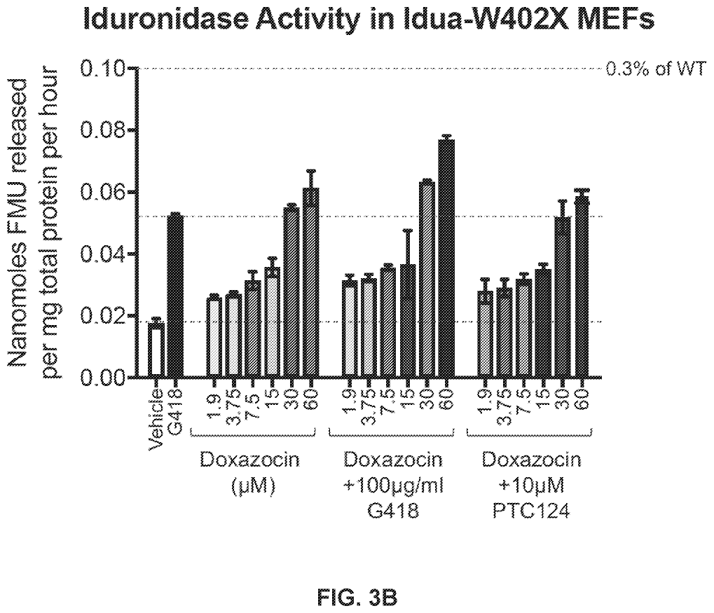

To further examine the function of the 15 hits from the initial screen, next determined was whether these compounds restored α-L-iduronidase activity in immortalized Idua-W402X MEFs (FIG. 2), which were generated from homozygous Idua-W402X mice that carry a PTC homologous to the 1DUA-W402X nonsense mutation, the most common mutation among MPS I-H patients (Wang, D., et al. (2010) Mol. Genet. Metab. 99:62-71, Gunn, G., et al. (2013) Mol. Genet. Metab. 111:374-381, Keeling, K. M., et al. (2013) PloS One 8:e60478). Among the 15 hits, three increased α-L-iduronidase activity above background levels. These included the FDA-approved drugs triamterene, doxazocin and vorinostat. Importantly, triamterene and doxazocin restored more α-L-iduronidase activity than the potent, but toxic, aminoglycoside G418. Further examination of triamterene (FIG. 3A) and doxazocin (FIG. 3B) showed that they both increased α-L-iduronidase activity in a dose-dependent manner. However, in contrast to the FRT NanoLuc reporter, synergy was not observe between triamterene (FIG. 3A) or doxazocin (FIG. 3B) with either G418 or PTC124 (a non-aminoglycoside readthrough agent) in Idua-W402X MEFs.

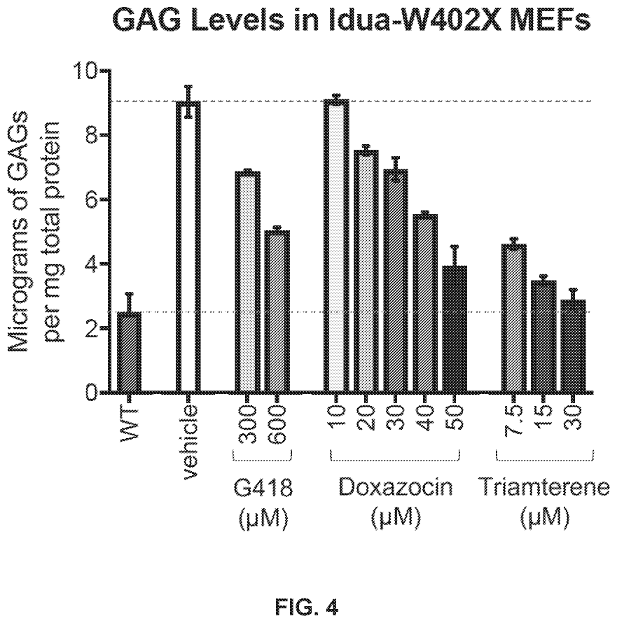

Next surveyed was whether triamterene or doxazocin restored enough α-L-iduronidase activity to produce a corresponding reduction of glycosaminoglycan (GAG) accumulation in Idua-W402X MEFs (Gunn, G., et al. (2013) Mol. Genet. Metab. 111:374-381; Keeling, K. M., et al. (2013) PloS One 8:e60478; Wang, D., et al, (2012) Mol. Genet. Metab. 105:116-125) (FIG. 4). Using a GAG dye-binding assay, a dose-dependent decrease in GAG accumulation was observed in Idua-W402X MEFs treated with doxazocin or triamterene, with triamterene reducing GAGs to near wildtype levels. This indicates that the α-L-iduronidase activity generated by triamterene or doxazocin is functional and that adequate levels of enzyme are restored to modulate downstream effectors.

Examination of Triamterene Mode of Action

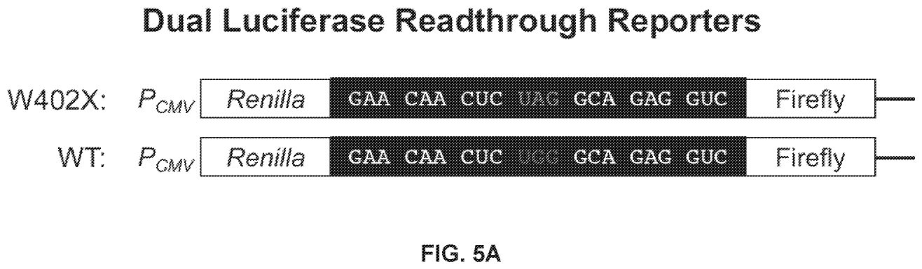

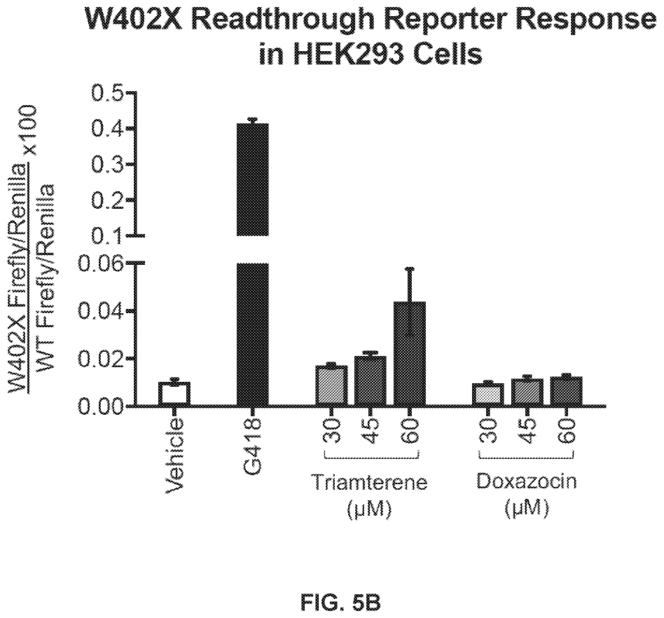

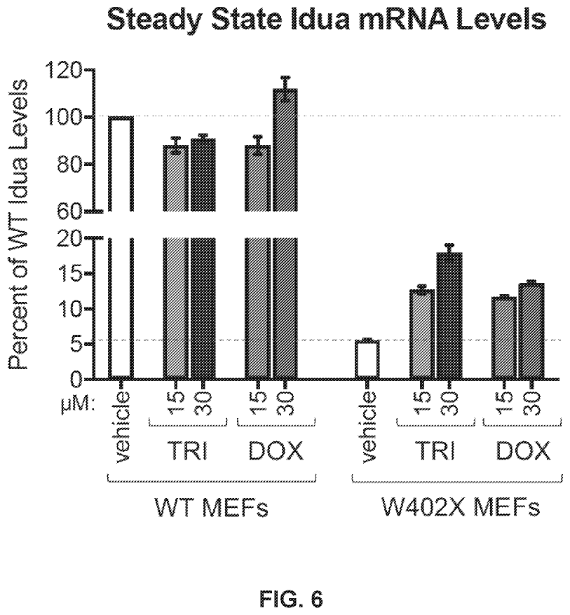

To determine whether triamterene generated α-L-iduronidase activity through readthrough of the Idua-W402X FTC, the W402X mutation (UAG) and three codons of upstream and downstream flanking Idua mRNA context were introduced into a dual luciferase readthrough reporter (Wang, D., et al. (2012) Mol. Genet. Metab. 105:116-125) (FIG. 5A). This reporter consists of an upstream Renilla luciferase and a downstream firefly luciferase separated by a readthrough cassette containing the PTC context. The percent readthrough of the PTC is measured by the amount of firefly activity generated relative to Renilla from the PTC construct relative to the WT construct×100. These reporters were stably expressed in HEK293 cells. In contrast to the Idua-W402X MEFs, triamterene was not as effective as G418 at suppressing the Idua-W402X FTC in HEK293 (FIG. 5B). However, a triamterene dose-dependent increase in Idua-W402X readthrough was still observed using the dual luciferase reporter, while no significant increase in readthrough relative to basal levels was detected with doxazocin. This suggests that triamterene has at least modest readthrough activity, while it was not confirmed whether doxazocin acts via a readthrough mechanism

Next examined was whether triamterene or doxazocin increase α-L-iduronidase activity by increasing Idua mRNA levels. Idua steady state mRNA was quantified in Idua-W402X or wildtype MEFs using qPCR (Keeling, K. M., et al. (2013) PloS One 8:e60478) (FIG. 5B). In wildtype MEFs, triamterene did not significantly change Idua mRNA levels, while doxazocin slightly increased Idua mRNA levels compared to the vehicle alone control. However, in Idua-W402X MEFs, which have much lower Idua steady state mRNA levels due to NMD, triamterene increased Idua mRNA levels 2.5- to 3.5-fold, while doxazocin increased Idua mRNA levels by ˜2-fold. These results suggest that triamterene suppresses the Idua-W402X mutation through a combination of readthrough and Idua mRNA stabilization, while doxazocin appears to work mainly by increasing Idua mRNA levels in a FTC-independent manner.

Next examined was whether triamterene affects α-L-iduronidase activity and GAG levels in immortalized MEFs derived from an MPS I-H knockout mouse (Idua-KO) that carries an insertional mutation in the mouse Idua locus that is not amenable to readthrough (Clarke, L. A., et al. (1997) Hum. Mol. Genet. 6:503-511; Russell, C., et al. (1998) Clin. Genet. 53:349-361). While slight increases in α-L-iduronidase in Idua-KO MEFs were observed, potentially as a result of alternative splicing, the amount of α-L-iduronidase restored in Idua-KO MEFs was far less than observed in Idua-W402X MEFs. GAG analysis results correlated well with the α-L-iduronidase assay, where a large decrease in GAGs was observed in triamterene-treated Idua-W402X MEFs, while little to no reduction in GAGs was observed in treated Idua-KO MEFs. These results further suggest that the mode of triamterene action to alleviate MPS I-H biochemical endpoints is through a FTC-dependent mechanism.

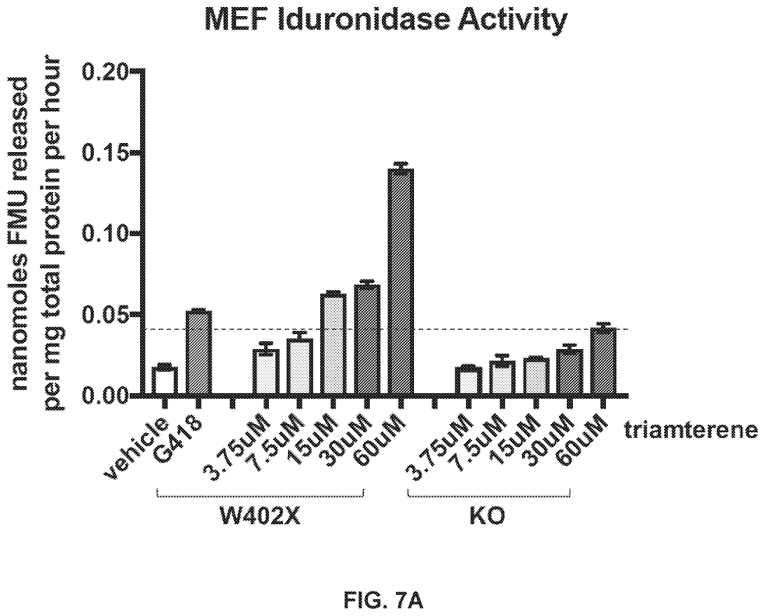

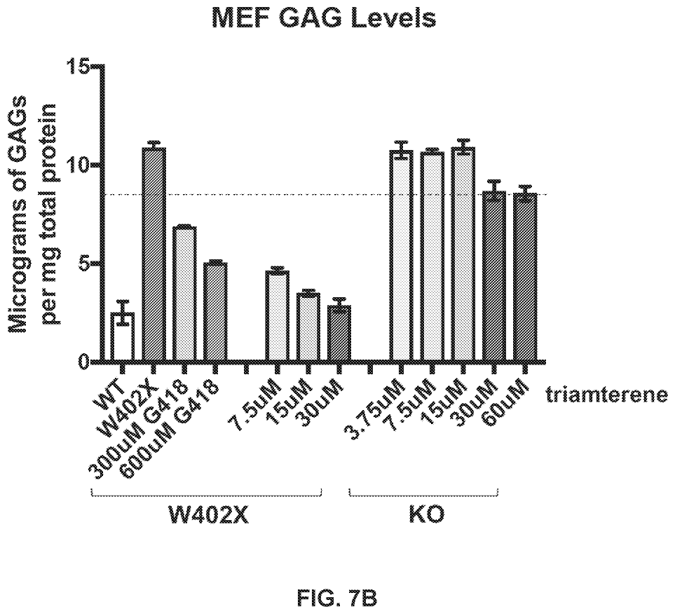

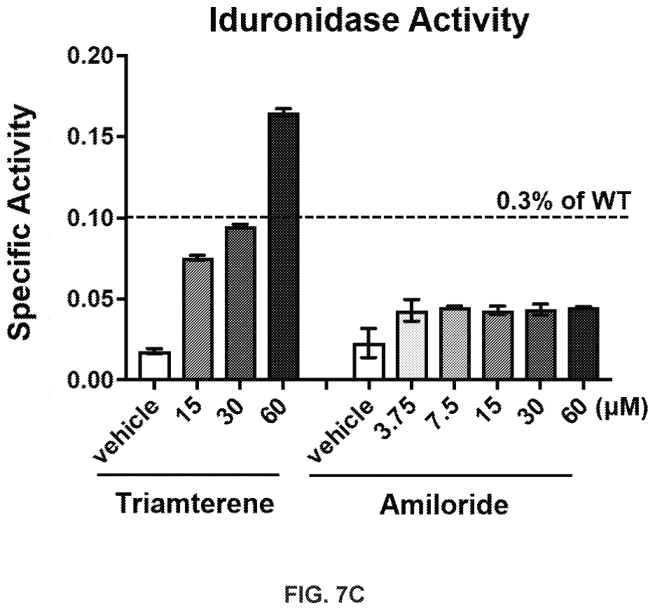

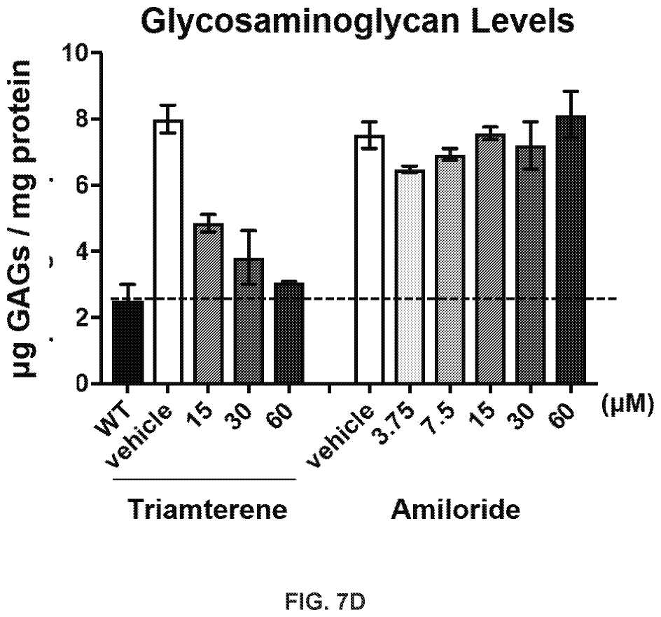

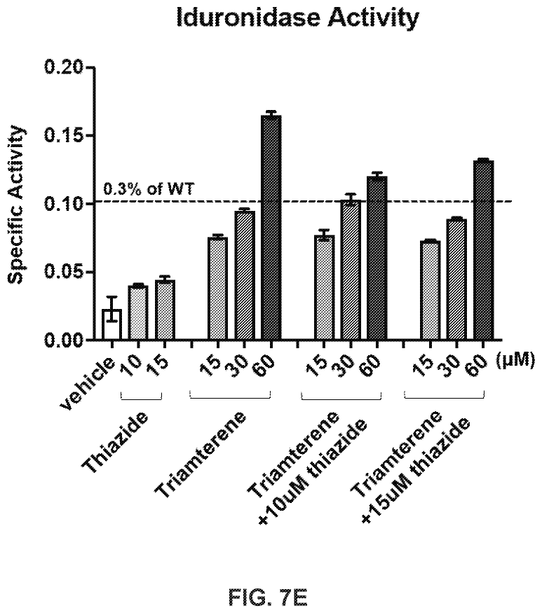

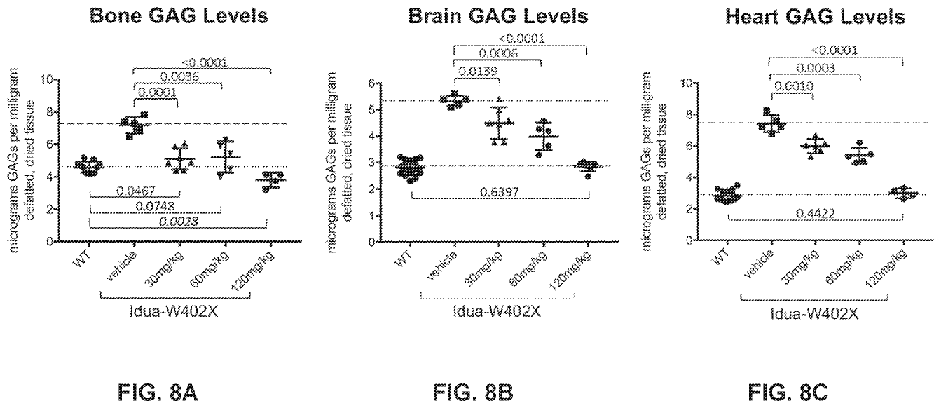

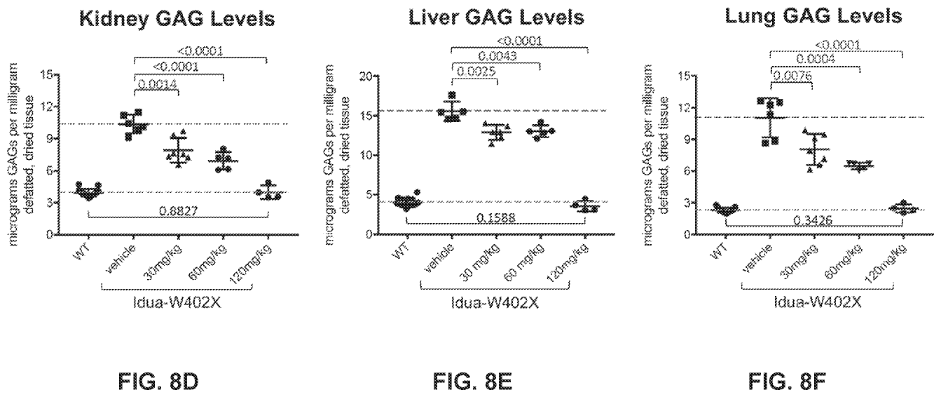

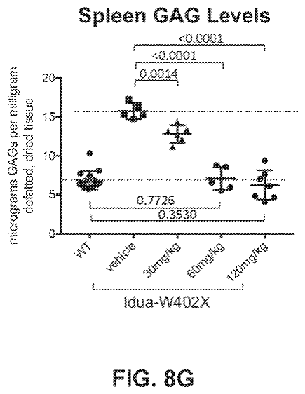

By blocking the epithelial sodium channel (ENaC) in the distal kidney, triamterene acts a potassium-sparing diuretic that is used to treat hypertension and edema. Another well-known ENaC inhibitor is amiloride, which has a chemical structure that is distinct from triamterene. We tested whether amiloride, via its ENaC-blocking function, also affected α-L-iduronidase activity and GAG levels in Idua-W402X MEFs. Unlike triamterene, amiloride did not generate a significant, dose-dependent increase in α-L-iduronidase activity (FIG. 7C). Furthermore, amiloride did not reduce GAGs in Idua-W402X MEFs (FIG. 7D). These data suggest that triamterene moderates MPS I-H biochemical endpoints through a mechanism that is independent from its role as an ENaC inhibitor. Analysis of thiazide, another diuretic that is clinically co-administered with triamterene and marketed as Dyazide™, did not affect αL-iduronidase activity or GAG levels in Idua-W402X MEFs (FIG. 7E). Furthermore, co-administration of thiazide with triamterene did not inhibit the ability of triamterene to relieve these MPS I-H biochemical endpoints.

In Vivo Administration of Triamterene Normalizes GAG Levels in Idua-W402X Mice

Next examined was whether triamterene normalized MPS I-H endpoints in vivo. Triamterene was administered to 8-week-old Idua-W402X mice via once daily oral gavage for a total of two weeks. At the end of treatment, GAG levels were measured in mouse tissues using a GAG dye-binding spectrophotometric assay (Gunn, G., et al. (2013) Mol. Genet. Metab. 111:374-381; Keeling, K. M., et al. (2013) PloS One 8:e60478; Wang, D., et al. (2012) Mol. Genet. Metab. 105:116-125). In all tissues examined, there was a triamterene dose-dependent decrease in GAGs in treated Idua-W402X mice relative to vehicle controls. At the 120 mg/kg dose, triamterene reduced GAGs to wildtype levels in all tissues examined. These results suggest that triamterene is also effective in alleviating MPS I-H biochemical endpoints in vivo and may be a potential treatment for MPS I-H patients who carry the Idua-W402X nonsense mutation.

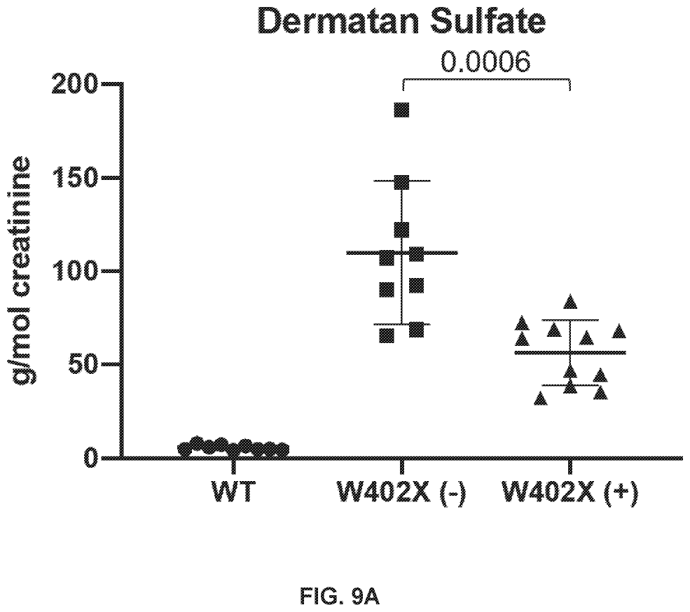

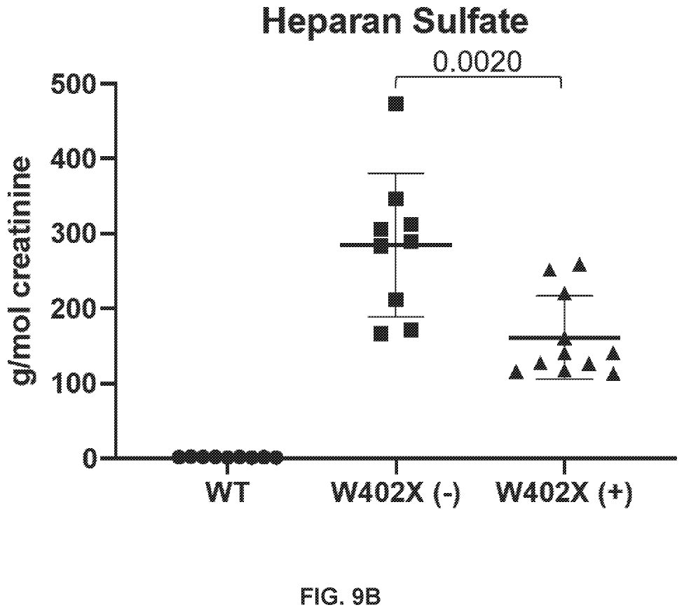

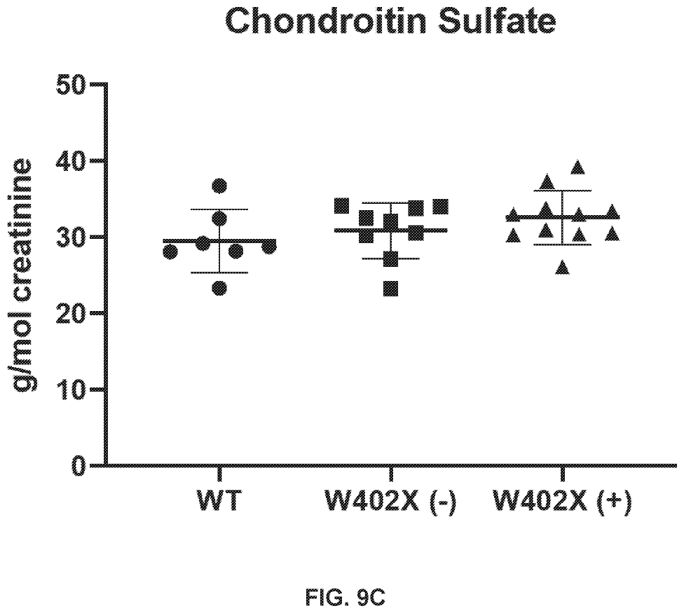

Also examined was whether GAGs levels were reduced in urine samples from Idua-W402X mice after triamterene administration. Idua-W402X mice were administered 120 mg/kg triamterene via once daily oral gavage for 2 weeks. Urine samples were collected at the end of treatment and subjected to mass spectrometry analysis to quantify the levels of dermatan sulfate (FIG. 9A), heparan sulfate (FIG. 9B), and chondroitin sulfate (FIG. 9C). Being substrates for α-L-iduronidase, both dermatan sulfate and heparan sulfate were significantly elevated in Idua-W402X mouse urine samples compared to wild-type controls. However, after completing two weeks of triamterene treatment, both dermatan and heparan sulfate were significantly decreased in Idua-W402X mouse urine compared to untreated controls. Chondroitin sulfate, a GAG that is not degraded by α-L-iduronidase, remained unchanged among all cohorts. These results indicate that only GAGs specifically targeted by α-L-iduronidase were altered, which is consistent with the restoration of α-L-iduronidase by readthrough of the Idua-W402X nonsense mutation.

TABLE 1
List of hits identified using a NanoLuc dual readthrough/NMD
reporter in the presence low dose G418.

| | | | | Drug | |
|---|---|---|---|---|---|
| | | | | Alone | +G418 |
| | | | | Fold- | Fold- |
| | | | | Increase | Increase |
| | | Drug | | (Rel. to | (Rel. to |
| Compound | Function | Conc. | | Basal) | G418) |
| Imantinib | Bcr-Abl inhibitor; anti-cancer | 60 | μM | 4.6 | 19.1 |
| Triamterene | Blocks ENaC; diuretic for | 60 | μM | 13.4 | 15.7 |
| | hypertension & edema | | | | |
| AB00993554 | SR compound | 30 | μg/ml | 7.1 | 10.6 |
| Doxazocin | a1-sensitive alpha blocker; | 60 | μM | 4.1 | 10.3 |
| | hypertension & urinary retention | | | | |
| GS-6201 | Adenosine A_{2B} antagonist; | 60 | μM | 10.3 | 13.6 |
| | anti-inflammatory | | | | |
| AB00990535 | SR compound | 30 | μg/ml | 12.3 | 8.5 |
| Verteporfin | Photosensitizer to eliminate | 60 | μM | 4.3 | 7.1 |
| | abnormal blood vessels in the eye | | | | |
| AB00989308 | SR compound | 30 | μg/ml | 6.5 | 7.1 |
| AB00993402 | SR compound | 30 | μg/ml | 10.1 | 6.8 |
| AB00989525 | SR compound | 30 | μg/ml | 5.7 | 7.1 |
| Tandutinib | FLT3 inhibitor; anti-cancer | 60 | μM | 3.8 | 7.8 |
| AB00990301 | SR compound | 30 | μg/ml | 6.7 | 6.2 |
| Tadalafil | PDE5 inhibitor; hypertension; | 60 | μM | 2.3 | 6.3 |
| | erectile dysfunction | | | | |
| Naloxonazine | μ-opioid receptor antagonist | 60 | μM | 2.7 | 5.8 |
| Elacridar | ABC transporter inhibitor; tumor | 60 | μM | 4.8 | 6.0 |
| | multidrug resistance | | | | |
| G418 alone | Readthrough control | 100 | μg/ml | 24 | — |

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the ark will recognize, or be able to ascertain using no more than routine experimentation, many equivalents to the specific embodiments of the invention described herein. Such equivalents are intended to be encompassed by the following claims.

What is claimed is:

1. A method for treating a genetic disorder in a subject, comprising administering to a subject determined to have a genetic disorder caused by a nonsense mutation a therapeutically effective amount of a composition comprising triamterene, or a pharmaceutically acceptable salt, hydrate, solvate, clathrate, prodrug, or polymorph thereof, in a pharmaceutically acceptable carrier.

2. The method of claim 1, wherein the subject has Hurler syndrome (mucopolysaccharidosis type IH (MPS IH)).

3. The method of claim 1, wherein the subject has cystic fibrosis.

4. The method of claim 1, wherein the subject has Duchenne muscular dystrophy.

5. A method for treating a genetic disorder in a subject, comprising
(a) assaying a sample from the subject for a nonsense mutation;
(b) detecting a nonsense mutation that causes a genetic disorder; and
(c) administering to the subject a therapeutically effective amount of a composition comprising triamterene, or a pharmaceutically acceptable salt, hydrate, solvate, clathrate, prodrug, or polymorph thereof, in a pharmaceutically acceptable carrier.

6. The method of claim 5, wherein a nonsense mutation that causes Hurler syndrome is detected.

7. The method of claim 5, wherein a nonsense mutation that causes cystic fibrosis is detected.

8. The method of claim 5, wherein a nonsense mutation that causes Duchenne muscular dystrophy is detected.


SEQUENCE LISTING (14 sequences)

SEQ ID NO: 1 - DNA, 56 bp, Artificial Sequence
  Synthetic Construct
tcgagccaagcttgcatgcctgcaggtcgactctagaggatccccggggaattcgc

SEQ ID NO: 2 - DNA, 56 bp, Artificial Sequence
  Synthetic Construct
ggccgcgaattccccggggatcctctagagtcgacctgcaggcatgcaagcttggc

SEQ ID NO: 3 - DNA, 21 bp, Artificial Sequence
  Synthetic Construct
gggaccctgtgaaacggcaac

SEQ ID NO: 4 - DNA, 21 bp, Artificial Sequence
  Synthetic Construct
gttgccgtttcacagggtccc

SEQ ID NO: 5 - DNA, 28 bp, Artificial Sequence
  Synthetic Construct
tcgacggaacaactctaggcagaggtcg

SEQ ID NO: 6 - DNA, 28 bp, Artificial Sequence
  Synthetic Construct
gatccgacctctgcctagagttgttccg

SEQ ID NO: 7 - DNA, 28 bp, Artificial Sequence
  Synthetic Construct
tcgacggaacaactctgggcagaggtcg

SEQ ID NO: 8 - DNA, 28 bp, Artificial Sequence
  Synthetic Construct
gatccgacctctgcccagagttgttccg

SEQ ID NO: 9 - DNA, 24 bp, Artificial Sequence
  Synthetic Construct
tgacaatgccttcctgagctacca

SEQ ID NO: 10 - DNA, 24 bp, Artificial Sequence
  Synthetic Construct
tgactgtgagtactggctttcgca

SEQ ID NO: 11 - DNA, 23 bp, Artificial Sequence
  Synthetic Construct
ttccagtatgactccactcacgg

SEQ ID NO: 12 - DNA, 25 bp, Artificial Sequence
  Synthetic Construct
tgaagacaccagtagactccacgac

SEQ ID NO: 13 - DNA, 20 bp, Artificial Sequence
  Synthetic Construct
atgacaagaaaaagcggatg

SEQ ID NO: 14 - DNA, 20 bp, Artificial Sequence
  Synthetic Construct
cttttctgcctgtttccgta